(19) United States
(12) Patent Application Publication
(10) Pub. No.: US 2017/0157409 A1
(43) Pub. Date: Jun. 8, 2017

(54) MEDICAL DEVICE REMOTE CONTROL CASE COVERS

(71) Applicants: Mitzi Gonzalez, San Antonio, TX (US); Narciso Gonzalez, San Antonio, TX (US)

(72) Inventors: Mitzi Gonzalez, San Antonio, TX (US); Narciso Gonzalez, San Antonio, TX (US)

(21) Appl. No.: 14/959,365

(22) Filed: Dec. 4, 2015

(51) Int. Cl. A61N 1/372; A61N 1/36

(52) U.S. Cl. CPC A61N 1/37247; A61N 1/36071

(57) ABSTRACT

A system to protect a neuro stimulator remote controller that includes: a cover, where the cover fits over a remote controller for a neuro stimulator; and openings over the surface of the cover, wherein the openings expose components of the remote controller. The components may include control buttons and at least one screen on the remote controller. Further, the present invention relates to a method of manufacturing a cover for a neuro stimulator remote controller that includes: defining any interactive components of a neuro stimulator remote controller; and creating a cover for the neuro stimulator remote controller, wherein the cover includes openings to gain access to the interactive components during use thereof. The interactive components may include a screen and control buttons.

### BACKGROUND OF THE INVENTION

Field of Invention

The present invention relates to a case cover that is used to cover a remote control that is used in conjunction with neuro stimulation devices.

Description of Related Art

Nerve/neuro stimulation devices are used to control pain as part of a pain management treatment plan. The electronic stimulators use an electrical signal delivered through electrodes through a patient's skin. These electrical signals are utilized to mask pain signals and therefore provide relief for chronic pain conditions for various patients. Many of these neuro stimulators include a remote control for the patient to utilize and generate various pulses as needed for pain relief. However, remotes currently used on various pain simulators are quite fragile and can become broken if dropped or mishandled by the user. Because many patients may have limited mobility issues may arise in the mishandling of the remote while under the stimulation care. As a result, it would be advantageous to have a cover to protect the remote during operation.

### SUMMARY OF THE INVENTION

The present invention relates to a system to protect a neuro stimulator remote controller that includes: a cover, where the cover fits over a remote controller for a neuro stimulator; and openings over the surface of the cover, wherein the openings expose components of the remote controller. The components may include control buttons and at least one screen on the remote controller. Further, the present invention relates to a method of manufacturing a cover for a neuro stimulator remote controller that includes: defining any interactive components of a neuro stimulator remote controller; and creating a cover for the neuro stimulator remote controller, wherein the cover includes openings to gain access to the interactive components during use thereof. The interactive components may include a screen and control buttons.

### BRIEF DESCRIPTION OF THE DRAWINGS

FIG. 1 depicts a neuro stimulator remote controller in accordance with the present invention.

FIG. 2 depicts a second neuro stimulator remote controller in accordance with the present invention.

### DETAILED DESCRIPTION

The present invention relates to covers that are provided for a remote controller used with neuro stimulation devices. The covers are provided for the remote controllers used with nerve stimulation devices therefore providing protection for the remote during use.

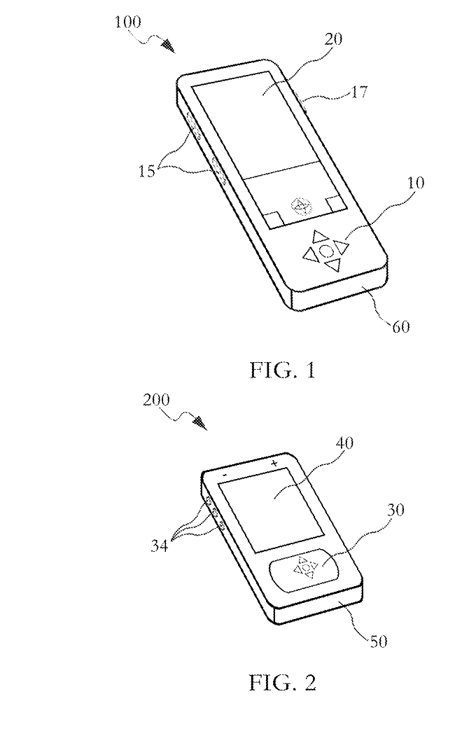

In reference to FIG. 1, a first remote controller 100 is depicted in accordance with the present invention. This first remote includes a screen 20 and control buttons 10 on the top surface of the remote 100. Further control buttons 10 are provided on the sides of the remote 100, control buttons 15 and control button 17 depicted on the opposing side. A cover 60 surrounds the entire remote 100 and protects the remote controller 100 from damage. As shown, this cover 60 has suitable openings to expose the screen 20, control buttons 15, 17 and 10, in order for a user to effectively use the remote but have the protection of the cover 60.

FIG. 2 depicts a second remote controller 100 in accordance with the present invention. This second remote controller 200 similarly has control buttons 30 on the top surface with a larger screen 40 provided. A series of control buttons 34 are provided on one side of this particular remote controller 200. Cover 50 covers and seals the remote controller 200 to protect it from damage during use. Again this cover 50 has suitable openings to expose control buttons 30, screen 40 and control buttons 34. The instant invention has been shown and described in what it considers to be the most practical and preferred embodiments. It is recognized, however, that departures may be made there from within the scope of the invention and that obvious modifications will occur to a person skilled in the art.

What is claimed is:

1. A system to protect a neuro stimulator remote controller comprising:
a. a cover, where the cover fits over a remote controller for a neuro stimulator; and
b. openings over the surface of the cover, wherein the openings expose components of the remote controller.

2. The system to protect a neuro stimulator according to claim 1, wherein the components include control buttons and at least one screen on the remote controller.

3. A method of manufacturing a cover for a neuro stimulator remote controller comprising:
a. defining any interactive components of a neuro stimulator remote controller; and
b. creating a cover for the neuro stimulator remote controller, wherein the cover includes openings to gain access to the interactive components during use thereof.

4. The method of manufacturing according to claim 3, where the interactive components include a screen and control buttons.
